(19) United States
(12) Patent Application Publication
(10) Pub. No.: US 2019/0062644 A1
(43) Pub. Date: Feb. 28, 2019

(54) PROCESSES FOR REFORMING AND TRANSALKYLATING HYDROCARBONS

(71) Applicant: UOP LLC, Des Plaines, IL (US)

(72) Inventors: Linda S. Cheng, Highland Park, IL (US); Ashish S. Bambal, Savannah, GA (US); Joseph A. Montalbano, Elmhurst, IL (US)

(21) Appl. No.: 15/692,480

(22) Filed: Aug. 31, 2017

(51) Int. Cl. C10G 29/20; C07C 7/04; C07C 5/27; C07C 6/12; B01J 8/08; B01J 8/02; B01J 29/06; B01J 29/80

(52) U.S. Cl. CPC C10G 29/205; C07C 7/04; C07C 5/2732; C07C 5/2708; C07C 6/126; B01J 2219/0004; B01J 8/02; B01J 29/061; B01J 29/80; C10G 2400/02; B01J 2219/00006; B01J 8/082

(57) ABSTRACT

Processes for reforming and transalkylating hydrocarbons are disclosed. A method for processing a hydrocarbon stream includes the steps of separating para-xylene from a first mixed-xylene and ethylbenzene-containing stream to produce a first non-equilibrium xylene and ethylbenzene stream and isomerizing the first non-equilibrium xylene and ethylbenzene stream to produce additional para-xylene. The method further includes transalkylating a toluene stream to produce a second mixed-xylene and ethylbenzene-containing stream, separating para-xylene from the second mixed-xylene and ethylbenzene-containing stream to produce a second non-equilibrium xylene and ethylbenzene stream, and isomerizing the second non-equilibrium xylene and ethylbenzene stream using a liquid phase isomerization process to produce additional para-xylene.

### FIELD

The technical field relates generally to hydrocarbon processing methods and systems. More particularly, the technical field relates to methods and systems for reforming and transalkylating hydrocarbons, such as naphtha hydrocarbons.

### BACKGROUND

The reforming of petroleum raw materials is an important process for producing useful products. One important process is the separation and upgrading of hydrocarbons for a motor fuel, such as producing a naphtha feedstream and upgrading the octane value of the naphtha in the production of gasoline. Furthermore, hydrocarbon feedstreams from a raw petroleum source include the production of useful chemical precursors for use in the production of plastics, detergents and other products.

Xylene isomers (“xylenes”) and benzene are produced in large volumes from petroleum by the reforming of naphtha. However, neither the xylenes nor benzene are produced in sufficient volume to meet demand. Consequently, other hydrocarbons are necessarily converted to increase the yield of the xylenes and benzene via processes such as transalkylation, disproportionation, isomerization, and dealkylation. For example, toluene commonly is disproportionated to yield benzene and C8 aromatics from which the individual xylene isomers are recovered.

In addition to xylene, some ethylbenzene is produced using the above-noted processes. However, there is a large difference in ethylbenzene (EB) concentration between mixed xylenes produced by transalkylation versus reforming. Modern transalkylation catalysts have a high dealkylation activity, which results in low yield of EB (about 1% of the C8 aromatic fraction). Reforming catalysts, in contrast, have low cracking activity and generate high concentrations of EB (about 14% of C8 aromatics).

In para-xylene purification processes, it is a requirement to convert EB to prevent accumulation in the recycle loops. Xylene isomerization processes may convert EB through either dealkylation or isomerization reactions. The dealkylation reaction converts EB to form benzene, while the isomerization reaction converts EB to xylene. EB isomerization is equilibrium limited reaction and requires increased hydraulic flow, increased energy consumption, and a high concentration of precious metals. The main advantage of EB isomerization is that it provides the highest yield of desirable para-xylene. EB dealkylation is not limited by equilibrium and therefore requires lower capital, lower energy, and much less precious metal. The drawback of EB dealkylation is that the para-xylene yield is much lower than EB isomerization.

Liquid phase isomerization is an attractive isomerization option; it isomerizes meta and ortho xylenes into an equilibrium xylene mixture; it is inexpensive; and it uses little utilities. But, its EB conversion is small. If a mixed xylene stream has a low EB concentration, liquid phase isomerization would be much desired.

In a typical aromatics complex, the mixed xylene produced by both isomerization processes and reforming processes are combined in a xylene splitter. The mixing of these two streams reduces the concentration of EB to a near equilibrium level. Because the feed is at equilibrium there is limited driving force to form xylene from EB using an EB isomerization process. Thus, the production of xylenes remains sub-optimal.

Accordingly, it is desirable to provide improved methods and systems for reforming and transalkylating hydrocarbons. It is further desirable to provide such methods and systems that are able to efficiently convert the produced ethylbenzene to xylenes. Furthermore, other desirable features and characteristics of the presently disclosed embodiments will become apparent from the subsequent detailed description and the appended claims, taken in conjunction with the accompanying drawings and this background.

### SUMMARY

Methods and systems for reforming and transalkylating hydrocarbons are disclosed. In one exemplary embodiment, a method for processing a hydrocarbon stream includes the steps of separating para-xylene from a first mixed-xylene and ethylbenzene-containing stream to produce a first non-equilibrium xylene and ethylbenzene stream and isomerizing the first non-equilibrium xylene and ethylbenzene stream to produce additional para-xylene. The method further includes transalkylating a toluene stream to produce a second mixed-xylene and ethylbenzene-containing stream, separating para-xylene from the second mixed-xylene and ethylbenzene-containing stream to produce a second non-equilibrium xylene and ethylbenzene stream, and isomerizing the second non-equilibrium xylene and ethylbenzene stream using a liquid phase isomerization process to produce additional para-xylene.

In another exemplary embodiment, a system for processing a hydrocarbon stream includes a first para-xylene separating unit that separates para-xylene from a first mixed-xylene and ethylbenzene-containing stream to produce a first non-equilibrium xylene and ethylbenzene stream and an isomerization unit that isomerizes the first non-equilibrium xylene and ethylbenzene stream to produce additional para-xylene. The system further includes a transalkylation unit that transalkylates a toluene stream and a C9+ aromatic streams to produce a second mixed-xylene and ethylbenzene-containing stream, a second para-xylene separating unit that separates para-xylene from the second mixed-xylene and ethylbenzene-containing stream to produce a second non-equilibrium xylene and ethylbenzene stream, and a liquid phase isomerization unit that dealkylates the second non-equilibrium xylene and ethylbenzene stream to produce additional para-xylene.

In the foregoing, all temperatures are set forth in degrees Celsius and, all parts and percentages are by weight, unless otherwise indicated. Other objects, advantages and applications of the present invention will become apparent to those skilled in the art from the following detailed description and drawing. Additional objects, advantages and novel features of the examples will be set forth in part in the description which follows, and in part will become apparent to those skilled in the art upon examination of the following description and the accompanying drawing or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWING

The FIGURE illustrates a schematic illustration of a process for reforming and transalkylating hydrocarbons, such as naphtha hydrocarbons, in accordance with exemplary embodiments.

### DETAILED DESCRIPTION

The following detailed description is merely exemplary in nature and is not intended to limit the application and uses of the embodiment described. Furthermore, there is no intention to be bound by any theory presented in the preceding background or the following detailed description.

Various embodiments contemplated herein relate to methods and systems for reforming and transalkylating hydrocarbons. Various values of temperature, pressure, flow rates, number of stages, feed entry stage number etc. are recited in association with the specific examples described below; those conditions are approximate and merely illustrative, and are not meant to limit the described embodiments. Additionally, for purposes of this disclosure, when the terms “middle”, “top” or “lower” are used with respect to a column, these terms are to be understood as relative to each other, i.e. that withdrawal of a stream from the “top” of the column is at a higher position than the stream withdrawn from a “lower” portion of the column. When the term “middle” is used it implies that the “middle” section is somewhere between the “upper” and the “lower” section of the column. However, when the terms “upper”, “middle” and “lower” have been used with respect to a distillation column it should not be understood that such a column is strictly divided into thirds by these terms.

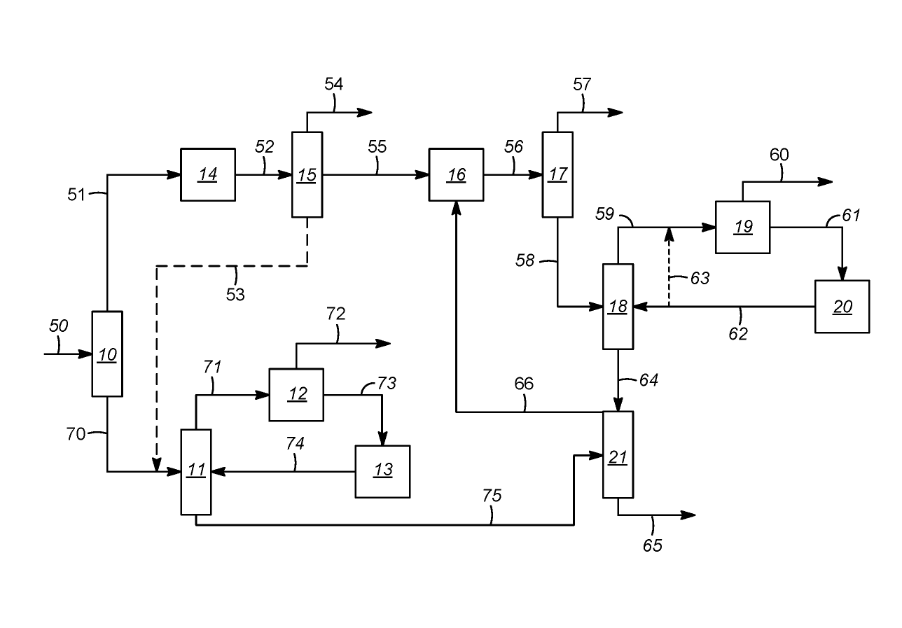

The description of the apparatus of this invention is presented with reference to the attached FIGURE. The FIG. 1s a simplified diagram of the preferred embodiment of this invention and is not intended as an undue limitation on the generally broad scope of the description provided herein and the appended claims. Certain hardware such as valves, pumps, compressors, heat exchangers, instrumentation and controls, have been omitted as not essential to a clear understanding of the invention. The use and application of this hardware is well within the skill of the art.

As shown in the FIGURE, a process comprises of a reformate stream 50 can be passed to a reformate splitter 10 to generate a reformate splitter overhead stream 51 including C₆ and C₇ aromatics, and a bottoms stream 70 including C₈ and heavier aromatics. The reformate overhead stream 51 is passed to an aromatics recovery unit 14 thereby generating an aromatics product stream 52 including benzene and toluene. The aromatics recovery unit may additionally generate a raffinate stream including paraffins. Aromatics product stream 52 may be further separated using either a single or a series of fractionation columns 15, wherein fractionation column(s) 15 receives product stream 52 and produces an overhead benzene product 54 and a bottoms A8+ product 53 and a side toluene product 55. The bottoms product 53 from fractionation column 15 may include xylenes, and as such may be recycled for xylene fractionation, as will be described in greater detail below.

Returning to the discussion of the reformate splitter 10, the splitter bottoms stream 70 proceeds to a first xylene fractionation column 11 that produces a xylene-containing hydrocarbon stream 71 as its overhead product, and heavier hydrocarbons as its bottoms product 75. As noted above, fractionation column 15 bottoms product stream 53 may join stream 70 in the first xylene fractionation column 11. As initially noted above, as a result of the reforming process, ethylbenzene is produced, in an amount that may be about 14% of the C₈ hydrocarbons produced.

Xylene-containing hydrocarbon stream 71 may thereafter be passed to a first para-xylene separation zone 12. Para-xylene is almost exclusively separated from xylene mixtures using simulated moving bed (SMB) technology. The SMB process is a commercial adsorptive separation process using several adsorption beds and moving the inlet streams and outlet streams between the beds, where a process stream comprising para-xylene is passed through the beds. The adsorption beds comprise an adsorbent for preferentially adsorbing the para-xylene and later desorbing the para-xylene using a desorbent, as the process stream. The SMB process may use a single adsorbent that has the best characteristics for preferentially adsorbing para-xylene, or multiple adsorbents.

It is preferred to operate the adsorption zone at conditions which include a temperature between about 120° C. and 200° C. (249° F. to 392° F.) as this provides better selectivity and capacity. Another important operational variable is the water content of the molecular sieve. This variable is necessary for mass transfer considerations, but there is a tradeoff in that water enhances mass transfer of the para-xylene, but reduces capacity of both the para-xylene and total C₈ aromatic capacity. Therefore, a balance must be achieved to optimize the process. As a commercial process operates continuously with the adsorbent confined within the chambers the acknowledged method of operation includes adding water, as required, to the feed stream. The level of hydration of the adsorbent is reported on a volatile free basis or by a measurement referred to as Loss on Ignition (LOI) as described in U.S. Pat. No. 5,900,523. In the LOI test the volatile matter content of the zeolitic adsorbent is determined by the weight difference obtained before and after drying a sample of the adsorbent at 900° C. under an inert gas purge such as nitrogen for a period of time sufficient to achieve a constant weight. Other operating variables include the L3/A ratio and the A/F ratio. The L3/A ratio is the ratio of liquid flow through zone 3 of the SMB system to the rate of simulated circulation of selective pore volume through the process. The A/F is the ratio the rate of simulated circulation of selective pore volume through the process to the volumetric feed rate of the feed mixture. The A/F ratio sets an operating curve, specific to a particular L3 rate. Operating with an A/F ratio of about 0.3 to about 0.7 is preferred. A process unit designed for normally producing a high purity product (e.g., 99%) will operate at the higher end of this general range.

Adsorbents known in the art include an X zeolite, and preferably the adsorbent is barium substituted X zeolite, or BaX. They also include a Y zeolite, and preferably the adsorbent is potassium substituted Y zeolite, or KY. X zeolites are known in the art for use in the separation of para-xylene as described in U.S. Pat. No. 6,706,938 and is incorporated by reference in its entirety. Y-zeolites are known in the art and are described in U.S. Pat. Nos. 4,842,836, 4,965,233, 6,616,899, and 6,869,521 and which are incorporated by reference in their entirety.

The separated para-xylene product from the first para-xylene separation zone 12 may be removed as stream 72. Some of the remaining xylenes and ethylbenzene may be passed to an isomerization zone 13 via stream 73. Stream 73 may include non-equilibrium xylenes and ethylbenzene. These aromatic compounds are in a non-equilibrium mixture, i.e., at least one C₈ aromatic isomer is present in a concentration that differs substantially from the equilibrium concentration at isomerization conditions. Thus, a non-equilibrium xylene composition exists where one or two of the xylene isomers are in less than equilibrium proportion with respect to the other xylene isomer or isomers. The xylene in less than equilibrium proportion may be the para-isomers, due to the previous separation thereof. Generally the mixture will have an ethylbenzene content of about 14 mass-% of C₈ compounds, an ortho-xylene content of 0 to about 35 mass-%, a meta-xylene content of about 20 to about 95 mass-%, and a para-xylene content of 0 to about 30 mass-%.

Especially advantageous as the xylene/ethylbenzene isomerization catalyst is a catalyst containing 12-membered rings and 10-membered rings in the same 3-dimensional structure. Commercial utility is typically seen in aluminosilicate structures which are synthesized in hydroxide media with readily available structure directing agents. Zeolites which contain both 12-membered and 10-membered rings in 3-dimensional structures belong to the CON, DFO, IWR, IWW and MSE structure types. The synthesis of CIT-1, a zeolite of the CON structure type, is described in U.S. Pat. No. 5,512,267 and in J. Am. Chem. Soc. 1995, 117, 3766-79 as a borosilicate form. After synthesis, a subsequent step can be undertaken to allow substitution of Al for B. The zeolites SSZ-26 and SSZ-33, also of the CON structure type are described in U.S. Pat. No. 4,910,006 and U.S. Pat. No. 4,963,337 respectively. SSZ-33 is also described as a borosilicate.

One particular zeolite of the MSE structure type, designated MCM-68, was disclosed by Calabro et al. in 1999 (U.S. Pat. No. 6,049,018). This patent describes the synthesis of MCM-68 from dication directing agents, N,N,N,N′-tetraalkylbicyclo[2.2.2]oct-7-ene-2R,3S:5R,6S-dipyrrolidinium dication, and N,N,N,N′-tetraalkylbicyclo[2.2.2]octane-2R,3S:5R,6S-dipyrrolidinium dication. MCM-68 was found to have at least one channel system in which each channel is defined by a 12-membered ring of tetrahedrally coordinated atoms and at least two further independent channel systems in which each channel is defined by a 10-membered ring of tetrahedrally coordinated atoms wherein the number of unique 10-membered ring channels is twice the number of 12-membered ring channels, see Patent Application Publication no. US 2009/318696 A1.

The non-equilibrium mixture, in the presence of hydrogen, is contacted with an isomerization catalyst as described above. Contacting may be effected using the catalyst system in a fixed-bed system, a moving-bed system, a fluidized-bed system, and an ebullated-bed system or in a batch-type operation. In view of the danger of attrition loss of valuable catalysts and of the simpler operation, it is preferred to use a fixed-bed system. In this system, the feed mixture is preheated by suitable heating means to the desired reaction temperature, such as by heat exchange with another stream if necessary, and then passed into an isomerization zone containing catalyst. The isomerization zone may be one or more separate reactors with suitable means therebetween to ensure that the desired isomerization temperature is maintained at the entrance to each zone. The reactants may be contacted with the catalyst bed in upward-, downward-, or radial-flow fashion.

The isomerization is conducted under isomerization conditions including isomerization temperatures generally within the range of about 100° to about 550° C. or more, and preferably in the range from about 150° to 500° C. The pressure generally is from about 10 kPa to about 5 MPa absolute, preferably from about 100 kPa to about 3 MPa absolute. The isomerization conditions comprise the presence of hydrogen in a hydrogen to hydrocarbon mole ratio of between about 0.5:1 to 6:1, preferably about 1:1 or 2:1 to 5:1. A sufficient mass of catalyst comprising the catalyst (calculated based upon the content of molecular sieve in the catalyst composite) is contained in the isomerization zone to provide a weight hourly space velocity with respect to the liquid feed stream (those components that are normally liquid at STP) of from about 0.1 to 50 hr⁻¹, and preferably 0.5 to 25 hr⁻¹.

The isomerization zone product stream 74 may thereafter be passed to the xylene fractionator 11. The bottoms stream 75 may be passed to a heavy aromatics column 22. As such, the isomerization zone isomerized the non-equilibrium xylenes and ethylbenzene to produce additional para-xylene, which is then recycled back to the xylene column 11 and then the heavy aromatics column 21 for additional separation and recovery of para-xylene, thereby increasing process yields.

The feed to the transalkylation/disproportionation zone 16 includes the toluene from stream 55, as well as C₉ aromatics that are obtained from the stream 66 after processing through a heavy aromatics column 21 (resulting in an enriched C₉ stream 66), as will be described in greater detail below. C₁₀₊ hydrocarbons may also be present.

The stream of toluene 55 and the stream enriched in C₉ aromatics 66, together with or separate from each other, are fed to the transalkylation/disproportionation reaction zone 16. The streams 55 and 66 of toluene and enriched in C₉ aromatics are subjected to the disproportionation and transalkylation reactions in the presence of hydrogen in the reaction zone 16. The reactions produce a product mixture including benzene, C₈ aromatics, and C₁₀₊ hydrocarbons. The reactions in the reaction zone 16 proceed in the presence of catalysts. The catalysts employed in the reaction zone 16 can be one or more catalysts known in the art for these purposes, for example, a metal-containing zeolite. Of the catalyst, the metal can be one or more selected from the group consisting of Bi, Mo, Fe, Co, Ni, Pt, Ag, Pd, Re and Au, and the zeolite can be one or more selected from the group consisting of Y-type zeolite, mordenite, β-zeolite and ZSM-type zeolite. During reactions, the pressure is 1.0-5.0 MPa, the temperature is 300-480° C., and hydrogen/hydrocarbon molar ratio is 1-10. The space velocity by weight is maintained at 0.5-10 h⁻¹. The products of the disproportionation and transalkylation reactions are removed from the reaction zone 16 via the stream 56.

Stream 56 is sent to a benzene fractionation column 17 having an overhead stream 57 comprising benzene and a bottoms stream 58 that is sent to a second xylene fractionation column 18. Xylene fractionation column produces an overhead stream 59 including mixed xylenes and ethylbenzene, and a bottoms product stream 64 including heavier aromatic hydrocarbons which is sent to the heavy aromatics column 21.

Returning to the discussion of stream 75, which derives from the bottoms product of the first xylene fractionation column 11, stream 75 joins like stream 64 (both being bottoms of a xylene fractionation column) and proceeds to the heavy aromatics column 21. Heavy aromatics column 21, as mentioned above, produces as an overhead stream 66 the enriched C₉ aromatics product and as a bottoms stream 65 a heavy aromatics product, which may be removed from the system for uses in other products and/or processes.

The overhead stream 59 of the second xylene fractionation column 18 proceeds to a second para-xylene separation zone 19. As with the second para-xylene separation zone 19, discussed above, para-xylene separation zone may operate according to known SMB technology, using like catalysts and like processing conditions. The para-xylene product is removed via stream 60. Stream 61 is sent from the second para-xylene separation zone 19 to a liquid phase isomerization unit 20.

As initially noted above, the ethylbenzene present as a fraction of C₈ compounds may be about 0.2 to 1% in this transalkylation portion. These aromatic compounds are in a non-equilibrium mixture, i.e., at least one C₈ aromatic isomer is present in a concentration that differs substantially from the equilibrium concentration at isomerization conditions. Thus, a non-equilibrium xylene composition exists where one or two of the xylene isomers are in less than equilibrium proportion with respect to the other xylene isomer or isomers. The xylene in less than equilibrium proportion may be the para-isomers, due to the previous separation thereof. Generally, the mixture will have an ethylbenzene content of about 0.2 to 1 mass-% of C₈ compounds, an ortho-xylene content of 0 to about 35 mass-%, a meta-xylene content of about 20 to about 95 mass-%, and a para-xylene content of 0 to about 30 mass-%.

The catalyst used in the liquid phase isomerization zone 20 comprises a zeolite based catalyst. The liquid phase isomerization zone 20 receives an ortho-xylene rich stream which can effectively isomerize the xylenes with a zeolite based catalyst. Since liquid phase isomerization unit is operated under liquid phase, and does not need hydrogen, there is saving in operating expenditures. The major benefit comes from the lowering of ring losses across the EB isomerization unit along with a para-xylene yield improvement.

The isomerization unit 162 may comprise a single reactor or two or more separate reactors with suitable means there between to ensure that the desired isomerization temperature is maintained at the entrance to each reactor. The reactants may be contacted with the catalyst bed in upward-, downward-, or radial-flow fashion.

The liquid phase reactor system consists of only a reactor system and heat exchange equipment. Hydrogen addition is not required, nor is any vapor compressor, vapor/gas separation equipment, or separate fractionation. As such, liquid phase isomerization is an inexpensive and effective isomerization process when EB feed concentrations are low. Operating temperatures may range from 200 to 500 C and pressures from 15 to 40 barg.

The non-equilibrium xylene mixture is contacted with a liquid phase isomerization type catalyst as described above. The liquid phase isomerization zone product stream 62 may thereafter be passed to the second xylene fractionation column 18. Additionally, some portion of stream 62 (typically 5 to 60%) may be recycled to the second para-xylene extraction zone 19 via line 63 directly, thus bypassing and minimizing the fractionation requirement of column 18. As such, the liquid phase isomerization zone isomerizes the non-equilibrium xylenes to produce additional para-xylene, which is then recycled back to the second xylene column 18 and to the second para-xylene separation zone 19 for additional separation and recovery of para-xylene, thereby increasing process yields.

Accordingly, in the present disclosure, after separation of para-xylene, the non-equilibrium mixture of xylenes and ethylbenzene are processed in two separate zones that are optimized based on the amount of ethylbenzene to be expected in the respective stream. Accordingly, the mixed xylenes and EB produced in the reforming portion are preferentially processed using an EB isomerization based process; at about 14% of C₈ aromatic compounds, the C₈ aromatics in reformate have the highest EB concentration, and processing this stream with an EB isomerization catalyst improves the overall yield to para-xylene. In contrast, the mixed xylenes produced in transalkylation portion, at about 1% of the C₈ aromatics compounds, are preferentially processed using a liquid phase isomerization based catalyst. Because this mixed xylene stream has very low concentration of EB, processing this stream with a high efficiency liquid phase isomerization process lowers utilities and capital costs. Thus, as opposed to combining the mixed xylene stream, which as noted above is sometimes done in the prior art, the utilities and investment cost of this “hybrid” complex are significantly reduced, with improved product yield.

In some embodiments, various functions described herein are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

While the invention has been described with what are presently considered the preferred embodiments, it is to be understood that the invention is not limited to the disclosed embodiments, but it is intended to cover various modifications and equivalent arrangements included within the scope of the appended claims.

### Specific Embodiments

While the following is described in conjunction with specific embodiments, it will be understood that this description is intended to illustrate and not limit the scope of the preceding description and the appended claims.

A first embodiment of the invention is a process for processing a hydrocarbon stream comprising the steps of separating para-xylene from the first mixed-xylene and ethylbenzene-containing stream to produce a first non-equilibrium xylene and ethylbenzene stream; isomerizing the first non-equilibrium xylene and ethylbenzene stream to produce additional para-xylene; transalkylating a toluene stream to produce a second mixed-xylene and ethylbenzene-containing stream; separating para-xylene from the second mixed-xylene and ethylbenzene-containing stream to produce a second non-equilibrium xylene and ethylbenzene stream; and isomerizing the second non-equilibrium xylene and ethylbenzene stream using a liquid phase isomerization process to produce additional para-xylene. An embodiment of the invention is one, any or all of prior embodiments in this paragraph up through the first embodiment in this paragraph, wherein the first mixed-xylene and ethylbenzene-containing stream comprises a greater proportion of ethylbenzene than does the second mixed-xylene and ethylbenzene-containing stream. An embodiment of the invention is one, any or all of prior embodiments in this paragraph up through the first embodiment in this paragraph, further comprising reforming a naphtha-containing hydrocarbon stream to produce the first mixed-xylene and ethylbenzene-containing stream and the toluene stream. An embodiment of the invention is one, any or all of prior embodiments in this paragraph up through the first embodiment in this paragraph, further comprising hydrotreating the naphtha-containing hydrocarbon stream. An embodiment of the invention is one, any or all of prior embodiments in this paragraph up through the first embodiment in this paragraph, wherein separating para-xylene is performed using simulated moving bed processes.

A second embodiment of the invention is a system for processing a hydrocarbon stream comprising a first para-xylene separating unit that separates para-xylene from a first mixed-xylene and ethylbenzene-containing stream to produce a first non-equilibrium xylene and ethylbenzene stream; an ethylbenzene isomerization unit that isomerizes the first non-equilibrium xylene and ethylbenzene stream to produce additional para-xylene; a transalkylation unit that transalkylates a toluene and C9+ aromatic streams to produce a second mixed-xylene and ethylbenzene-containing stream; a second para-xylene separating unit that separates para-xylene from the second mixed-xylene and ethylbenzene-containing stream to produce a second non-equilibrium xylene and ethylbenzene stream; and a liquid phase isomerization unit that dealkylates the second non-equilibrium xylene and ethylbenzene stream to produce additional para-xylene. An embodiment of the invention is one, any or all of prior embodiments in this paragraph up through the second embodiment in this paragraph, wherein the first mixed-xylene and ethylbenzene-containing stream comprises a greater proportion of ethylbenzene than does the second mixed-xylene and ethylbenzene-containing stream. An embodiment of the invention is one, any or all of prior embodiments in this paragraph up through the second embodiment in this paragraph, further comprising a naphtha reforming unit that reforms a naphtha-containing hydrocarbon stream to produce the first mixed-xylene and ethylbenzene-containing stream and the toluene stream; An embodiment of the invention is one, any or all of prior embodiments in this paragraph up through the second embodiment in this paragraph, further comprising a naphtha hydrotreating unit that hydrotreates the naphtha-containing hydrocarbon stream. An embodiment of the invention is one, any or all of prior embodiments in this paragraph up through the second embodiment in this paragraph, wherein one or both of the first and second para-xylene separating units comprises a simulated moving bed.

A third embodiment of the invention is a process for processing a naphtha-containing hydrocarbon stream comprising the steps of hydrotreating a naphtha-containing hydrocarbon stream; reforming the naphtha-containing hydrocarbon stream to produce a first mixed-xylene and ethylbenzene-containing stream and a toluene stream, wherein the first mixed-xylene and ethylbenzene-containing stream comprises a greater proportion of ethylbenzene than does the second mixed-xylene and ethylbenzene-containing stream, and wherein the toluene stream is produced using aromatics recovery processes; separating para-xylene from the first mixed-xylene and ethylbenzene-containing stream to produce a first non-equilibrium xylene and ethylbenzene stream, wherein separating para-xylene is performed using simulated moving bed processes; isomerizing the first non-equilibrium xylene and ethylbenzene stream using ethylbenzene isomerization to produce additional para-xylene; transalkylating the toluene stream to produce a second mixed-xylene and ethylbenzene-containing stream; separating para-xylene from the second mixed-xylene and ethylbenzene-containing stream to produce a second non-equilibrium xylene and ethylbenzene stream, wherein separating para-xylene is performed using simulated moving bed processes; and isomerizing the second non-equilibrium xylene and ethylbenzene stream using a liquid phase isomerization process to produce additional para-xylene. An embodiment of the invention is one, any or all of prior embodiments in this paragraph up through the third embodiment in this paragraph, wherein hydrotreating comprises removing sulfur species. An embodiment of the invention is one, any or all of prior embodiments in this paragraph up through the third embodiment in this paragraph, wherein hydrotreating further comprises removing nitrogen species. An embodiment of the invention is one, any or all of prior embodiments in this paragraph up through the third embodiment in this paragraph, wherein the toluene stream is further produced using a first distillation column that removes benzene as an overhead product. An embodiment of the invention is one, any or all of prior embodiments in this paragraph up through the third embodiment in this paragraph, wherein the toluene stream is further produced using a second distillation column that removes toluene as an overhead product. An embodiment of the invention is one, any or all of prior embodiments in this paragraph up through the third embodiment in this paragraph, wherein transalkylating is performed in the presence of C9+ hydrocarbon species. An embodiment of the invention is one, any or all of prior embodiments in this paragraph up through the third embodiment in this paragraph, wherein transalkylating is further performed in the presence of a transalkylation catalyst. An embodiment of the invention is one, any or all of prior embodiments in this paragraph up through the third embodiment in this paragraph, isomerizing the first non-equilibrium xylene and ethylbenzene stream is performed in the presence of an isomerization catalyst. An embodiment of the invention is one, any or all of prior embodiments in this paragraph up through the third embodiment in this paragraph, wherein the naphtha-containing hydrocarbon stream is derived from a plurality of naphtha sources. An embodiment of the invention is one, any or all of prior embodiments in this paragraph up through the third embodiment in this paragraph, wherein the simulated moving bed processes are performed in the presence of a para-xylene adsorbent material.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for processing a hydrocarbon stream comprising the steps of:
separating para-xylene from the first mixed-xylene and ethylbenzene-containing stream to produce a first non-equilibrium xylene and ethylbenzene stream;
isomerizing the first non-equilibrium xylene and ethylbenzene stream to produce additional para-xylene;
transalkylating a toluene stream to produce a second mixed-xylene and ethylbenzene-containing stream;
separating para-xylene from the second mixed-xylene and ethylbenzene-containing stream to produce a second non-equilibrium xylene and ethylbenzene stream; and
isomerizing the second non-equilibrium xylene and ethylbenzene stream using a liquid phase isomerization process to produce additional para-xylene.

2. The process of claim 1, wherein the first mixed-xylene and ethylbenzene-containing stream comprises a greater proportion of ethylbenzene than does the second mixed-xylene and ethylbenzene-containing stream.

3. The process of claim 1, further comprising reforming a naphtha-containing hydrocarbon stream to produce the first mixed-xylene and ethylbenzene-containing stream and the toluene stream.

4. The process of claim 3, further comprising hydrotreating the naphtha-containing hydrocarbon stream.

5. The process of claim 1, wherein separating para-xylene is performed using simulated moving bed processes.

6. A system for processing a hydrocarbon stream comprising:
a first para-xylene separating unit that separates para-xylene from a first mixed-xylene and ethylbenzene-containing stream to produce a first non-equilibrium xylene and ethylbenzene stream;
an ethylbenzene isomerization unit that isomerizes the first non-equilibrium xylene and ethylbenzene stream to produce additional para-xylene;
a transalkylation unit that transalkylates a toluene and C9+ aromatic streams to produce a second mixed-xylene and ethylbenzene-containing stream;
a second para-xylene separating unit that separates para-xylene from the second mixed-xylene and ethylbenzene-containing stream to produce a second non-equilibrium xylene and ethylbenzene stream; and
a liquid phase isomerization unit that dealkylates the second non-equilibrium xylene and ethylbenzene stream to produce additional para-xylene.

7. The system of claim 6, wherein the first mixed-xylene and ethylbenzene-containing stream comprises a greater proportion of ethylbenzene than does the second mixed-xylene and ethylbenzene-containing stream.

8. The system of claim 6, further comprising a naphtha reforming unit that reforms a naphtha-containing hydrocarbon stream to produce the first mixed-xylene and ethylbenzene-containing stream and the toluene stream;

9. The system of claim 8, further comprising a naphtha hydrotreating unit that hydrotreates the naphtha-containing hydrocarbon stream.

10. The system of claim 6, wherein one or both of the first and second para-xylene separating units comprises a simulated moving bed.

11. A process for processing a naphtha-containing hydrocarbon stream comprising the steps of:
hydrotreating a naphtha-containing hydrocarbon stream;
reforming the naphtha-containing hydrocarbon stream to produce a first mixed-xylene and ethylbenzene-containing stream and a toluene stream, and wherein the toluene stream is produced using aromatics recovery processes;
separating para-xylene from the first mixed-xylene and ethylbenzene-containing stream to produce a first non-equilibrium xylene and ethylbenzene stream, wherein separating para-xylene is performed using simulated moving bed processes;
isomerizing the first non-equilibrium xylene and ethylbenzene stream using ethylbenzene isomerization to produce additional para-xylene;
transalkylating the toluene stream to produce a second mixed-xylene and ethylbenzene-containing stream, wherein the first mixed-xylene and ethylbenzene-containing stream comprises a greater proportion of ethylbenzene than does the second mixed-xylene and ethylbenzene-containing stream;
separating para-xylene from the second mixed-xylene and ethylbenzene-containing stream to produce a second non-equilibrium xylene and ethylbenzene stream, wherein separating para-xylene is performed using simulated moving bed processes; and
isomerizing the second non-equilibrium xylene and ethylbenzene stream using a liquid phase isomerization process to produce additional para-xylene.

12. The process of claim 11, wherein hydrotreating comprises removing sulfur species.

13. The process of claim 12, wherein hydrotreating further comprises removing nitrogen species.

14. The process of claim 11, wherein the toluene stream is further produced using a first distillation column that removes benzene as an overhead product.

15. The process of claim 14, wherein the toluene stream is further produced using a second distillation column that removes toluene as an overhead product.

16. The process of claim 11, wherein transalkylating is performed in the presence of C9+ hydrocarbon species.

17. The process of claim 16, wherein transalkylating is further performed in the presence of a transalkylation catalyst.

18. The process of claim 11, isomerizing the first non-equilibrium xylene and ethylbenzene stream is performed in the presence of an isomerization catalyst.

19. The process of claim 11, wherein the naphtha-containing hydrocarbon stream is derived from a plurality of naphtha sources.

20. The process of claim 11, wherein the simulated moving bed processes are performed in the presence of a para-xylene adsorbent material.
